(19) United States
(12) Patent Application Publication
(10) Pub. No.: US 2023/0346656 A1
(43) Pub. Date: Nov. 2, 2023

(54) EYEBROW PRIMER

(71) Applicant: L'ORÉAL, Paris (FR)

(72) Inventor: Sylvie PORET-FRISTOT, Clark, NJ (US)

(73) Assignee: L'ORÉAL, Paris (FR)

(21) Appl. No.: 17/732,697

(22) Filed: Apr. 29, 2022

(51) Int. Cl. A61K 8/25; A61K 8/81; A61Q 1/10; A61K 8/34

(52) U.S. Cl. CPC A61K 8/25; A61K 8/8182; A61Q 1/10; A61K 8/345; A61K 2800/48; A61K 2800/30

(57) ABSTRACT

A composition of matter that may be used as an eyebrow primer, and a method for using the composition of matter, are provided. The composition of matter may include at least 5% by weight of a transparent filler, a polyacrylamide thickener, at least 2% by weight of one or more high HLB surfactants, and a water-dispersed latex polymer. The composition of matter may be substantially free of oils.

### TECHNICAL FIELD

The present disclosure is drawn to cosmetic formulations and methods of use, and eyebrow primers in particular.

### BACKGROUND

Consumers may use eyebrow gels to enhance characteristics of their eyebrows. However, conventional eyebrow gels become dry and flakey even after less than a day's wear, making their brow feel stiff or crunchy. To provide the desired wear lifetime while also providing strong hold, and maintaining ease of application, a new approach is needed.

### BRIEF SUMMARY

In some embodiments, to provide the desired wear, hold, and ease of application, an eyebrow primer may be utilized. The eyebrow primer may include at least 5% by weight of a transparent filler, a polyacrylamide thickener, at least 2% by weight of one or more high HLB surfactants, and a water-dispersed latex polymer. In preferred embodiments, the eyebrow primer may be substantially free of oils. In some embodiments, the eyebrow primer may be free of pigments. In some embodiments, the eyebrow primer may be free of clays, such as hectorite.

In some embodiments, the transparent filler may include talc, mica, silica, powders of polyamide, poly-β-3-alanine powders; polyethylene powders; polyurethane powders, lauroyl lysine; starch; boron nitride; polymeric hollow microspheres, silicone resin powders, hollow silica microspheres; glass microcapsules; ceramic microcapsules; or a combination thereof. In some embodiments, the transparent filler may only include talc. In some embodiments, the eyebrow primer comprises 5%40% of talc.

In some embodiments, the one or more high HLB surfactants may include ceteareth-25. In some embodiments, the eyebrow primer may include at least 0.5% by weight of the polyacrylamide thickener. In some embodiments, the eyebrow primer may include at least 2% by weight of the water-dispersed latex polymer.

In some embodiments, the eyebrow primer may include a water-soluble polymer, such as polyvinylpyrrolidone (PVP). In some embodiments, the eyebrow primer may include at least 3% by weight of the water-soluble polymer.

In some embodiments, the eyebrow primer may include one or more C3-C6 polyhydric alcohols. In some embodiments, the eyebrow primer may include one or more additional surfactants or co-emulsifiers, the one or more additional surfactants or co-emulsifiers not being a high HLB surfactant. In some embodiments, the one or more additional surfactants or co-emulsifiers may be present in an amount of 1%-3% by weight of the eyebrow primer.

In some embodiments, the eyebrow primer may include a water-soluble humectant or solvent. In some embodiments, the water-soluble humectant or solvent may be present in an amount of 5%40% by weight of the eyebrow primer.

In some embodiments, a method for styling eyebrows is provided. The method may include applying an embodiment of an eyebrow primer as disclosed herein to an eyebrow, and then applying an eyebrow makeup composition, such as a pigmented eyebrow makeup composition, over the eyebrow primer.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “at least one” means one or more and thus includes individual components as well as mixtures/combinations.

As used herein, the term “between [two numbers]” is intended to include those two numbers. For example, “x is between 1 and 2” is intended to cover 1≤x≤2. The term “x is between about 1 and about 2” would be intended to cover 0.95≤x≤2.05.

As used herein, the term “free [of an ingredient]” means that the composition contains no detectable amounts of the identified ingredient. As used herein, the term “substantially free [of an ingredient]” means that the composition contains less than 1% of the identified ingredient.

As used herein, the terms “include”, “includes” and “including” are meant to be non-limiting.

All concentrations are by weight percent on an active basis unless otherwise indicated.

Eyebrow Primer

In some embodiments, an eyebrow primer is provided. The eyebrow primer is preferably an aqueous formula.

In some embodiments, the eyebrow primer includes water from 40%, 50%, or 60% up to about 70%, 80%, or 90% by weight of the eyebrow primer, including all combinations and subranges thereof. In some embodiments, the eyebrow primer comprises at least 60% by weight of water.

In some embodiments, the eyebrow primer may be an emulsion. However, any oil phase in the emulsion is preferably very small. Preferably, the eyebrow primer is substantially free of oils.

In some embodiments, the eyebrow filler may comprise, or consist of, water, a transparent filler, a high HLB surfactant, a polyacrylamide thickener, a water-dispersed latex polymer, a water-soluble polymer, a polyhydric alcohol, an additional surfactant or co-emulsifier, a water-soluble humectant and/or solvent, a chelating agent, a preservative, and an oil.

Transparent Filler

In some embodiments, the eyebrow primer may include at least 5% by weight of a transparent filler. Transparent fillers are generally those filler materials that, once applied to the skin and allowed to dry, does not leave a white (or colored) deposit. In some embodiments, the transparent filler may include talc, mica, silica, powders of polyamide, poly-β-3-alanine powders; polyethylene powders; polyurethane powders, lauroyl lysine; starch; boron nitride; polymeric hollow microspheres, silicone resin powders, hollow silica microspheres; glass microcapsules; ceramic microcapsules; or a combination thereof.

In some embodiments, the transparent filler may be talc.

The transparent filler, such as talc, may be coated or uncoated. A non-limiting example of a talc appropriate for the disclosed composition is that sold under the trade name Talc P-3® by the company Nippon Talc. The transparent filler used may have a particular size. While filler may (or may not) be spherical in shape, particle size analyzers are often used to provide a size estimate. In such cases, the filler may be found to have a median diameter (D50)≤7 microns, ≤5 microns, ≤3 microns, or ≤2 microns, and generally the D50 is ≥0.1 microns, ≥1 micron, or ≥1.5 microns, and/or any combination thereof.

In some embodiments, the total amount of transparent filler used in the eyebrow filler may be at least 5%, or at least 10% by weight of the eyebrow filler. In some embodiments, the eyebrow filler may include between 5% and 10% by weight of transparent filler.

High HLB Surfactants

The eyebrow primer may include one or more high HLB surfactants. As used herein, the term “high HLB surfactant” is intended to include any surfactant with a hydrophilic-lipophilic balance (HLB) greater than or equal to 9. In some embodiments, all high HLB surfactants have HLB values ≥10. In some embodiments, all the high HLB surfactants have HLB values ≥11. In some embodiments, the weighted HLB of all the high HLB surfactants is ≥11 (for example, if the composition comprises 1% of a 9 HLB surfactant and 4% of a 15 HLB surfactant, the weighted HLB would be 13.8). In some embodiments, the HLB of each high HLB surfactant is ≥15. The HLB value can be determined according to GRIFFIN in J. Soc. Cosm. Chem. 1954 (volume 5), pages 249-256.

In some embodiments, the compositions may include at least two high HLB surfactants. In some embodiments, the compositions may include at least three high HLB surfactants. In some embodiments, the compositions may include at least four high HLB surfactants.

In some embodiments, the high HLB surfactants may be fatty alcohols and/or fatty esters of polyethylene glycol (PEG) or glycerol.

As used herein, the term “fatty alcohol” includes an alcohol having an aliphatic tail, typically from 4 to 30 carbon atoms long. Fatty alcohols can be saturated, mono-unsaturated, polyunsaturated, linear or branched. Examples of fatty alcohols useful in the present invention include, but are not limited to, lauryl alcohol (C12), tetradecanol (C14), pentadecanol (C15), cetyl alcohol (C16), stearyl alcohol (C18), oleyl alcohol (C18), eicosanol (C20) and behenyl alcohol (C22). One of skill in the art will appreciate that other fatty alcohols are useful in the present invention.

As used herein, the term “fatty ester” includes any ester made from a fatty acid to produce, for example, a fatty acid ester. In one embodiment, a fatty ester contains an A side (i.e., the carbon chain attached to the carboxylate oxygen) and a B side (i.e., the carbon chain comprising the parent carboxylate). In a preferred embodiment, the A side is contributed by an alcohol, and the B side is contributed by a fatty acid. Any alcohol can be used to form the A side of the fatty esters.

As used herein, the term “fatty acid” means a carboxylic acid having the formula RCOOH. R represents an aliphatic group, preferably an alkyl group. R will generally comprise between about 4 and about 22 carbon atoms. Fatty acids can be saturated, monounsaturated, or polyunsaturated.

These high HLB surfactants can be selected from non-ionic, anionic, cationic and amphoteric surfactant agents. Preferably the surfactants are non-ionic.

Suitable surfactants of HLB greater than or equal to 9, used alone or as a mixture, include fatty alcohols or fatty esters.

In some embodiments, the high HLB surfactant may include a fatty alcohol such as cetyl, stearyl, and/or behenyl alcohol. In some embodiments, the high HLB surfactants may include cetyl alcohol and stearyl alcohol and may be cetearyl alcohol.

In some embodiments, the fatty alcohol may be oxyethylenated. In some embodiments, the fatty alcohol may include one or more oxyethylenated fatty alcohols with a number of oxyethylene units of greater than or equal to 10. In some embodiments, the fatty alcohol may include from 12 to 30 carbon atoms, such as 14 to 24 carbon atoms, such as 16 to 22 carbon atoms. In some embodiments, the number of oxyethylene units is preferably greater than or equal to 15. In some embodiments, the fatty alcohol may include oxyethylenated cetyl and stearyl alcohols, comprising at least 10, at least 15, or at least 20 oxyethylene units. In some embodiments, the fatty alcohol may include ceteareth-25 (mixture of cetyl and stearyl alcohols containing 25 oxyethylene units),

In some embodiments, the high HLB surfactant may include a fatty ester. In some embodiments, the fatty ester may include a polyethylene glycol, an ethoxylated and/or propoxylated ethers of glycerol, or an alkoxylated sorbitol.

In some embodiments, the high HLB surfactant may include a C8-C24, and preferably C16-C22, and polyethylene glycol (or PEG) (which can contain from 1 to 150 ethylene oxide units), such as the PEG-50 stearate or PEG-40 monostearate marketed under the MYRJ® brand name by Croda International.

In some embodiments, the high HLB surfactant may include a C8-C24, and preferably C16-C22, and ethoxylated and/or propoxylated ethers of glycerol (which can contain from 1 to 150 ethylene oxide and/or propylene oxide units), such as the polyglyceryl-10 dipalmitate or polyglyceryl-6 distearate. In some embodiments, the high HLB surfactant comprises a fatty ester that consists of one or more diesters of stearic acid.

In some embodiments, the high HLB surfactant may include an alkoxylated sorbitol, and particularly ethoxylataed sorbitol, such as polyoxyethylene (20) sorbitan monolaurate (alka referred to as polysorbate 20), polyoxyethylene (20) sorbitan monostearate (also referred to as polysorbate 60), or polyoxyethylene (20) sorbitan monooleate (also referred to as polysorbate 80).

In some embodiments, the high HLB surfactants may be free, or substantially free, of fatty alcohols. In some embodiments, the high HLB surfactants may be free, or substantially free, of fatty esters.

In some embodiments, the total amount of high HLB surfactant present in the eyebrow primer may be from at least 2%, 4%, or 6% up to 8%, 10%, or 12% by weight of the eyebrow primer, including all combinations and subranges thereof. In some embodiments, the high HLB surfactants may be present in a total amount from about 3% to about 8% by weight of the eyebrow primer.

Polyacrylamide Thickener

In some embodiments, the eyebrow primer may include a polyacrylamide thickener. As used herein, the term “polyacrylamide thickener” refers to polymers based upon one or more acrylamide monomers or similar monomers.

Non-limiting examples of suitable thickening agents include crosslinked anionic copolymers of acrylamide and of AMPS, e.g. in the form of a water-in-oil emulsion, such as those sold under the name SEPIGEL 305 (INCI name: Polyacrylamide/C13-14 lsoparaffin/Laureth-7) and under the name SIMULGEL 600 (CTFA name: Acrylamide/Sodium acryloyldimethyltaurate copolymer/Isohexadecane/Polysorbate 80); and those under the name SEPIPLUS 265 (Acrylamide/ammonium acrylate copolymer/polyisobutene/polysorbate 20), and those under the name SEPIMAX ZEN (ammonium 2-acrylamido 2-methylpropanesulfonate/dimethylacrylamide/hydrophobic chain copolymer, INCI: Polyacrylate Crosspolymer-6), all by SEPPIC. Other examples include those under the name SENSOGEL 200 (INCI: Hydroxyethyl acrylate/Sodium Acryloyl Dimethyl Taurate Copolymer by Applechem, Inc; those under the name Bluevisc AC (INCI: Acrylamide/Sodium acrylate Copolymer) by Blue Sun International; those under the name OPULYN 303B (INCI: Styrene Acrylamide Copolymer) by Univar Solutions; and those under the name Noevender EC-1 (INCI: Acrylates/Acrylamide/Copolymer) by Lubrizol.

In some embodiments, the polyacrylamide thickener may include an acrylamide/sodium acryloyldimethyltaurate copolymer.

In some embodiments, the eyebrow primer may include a total amount of the polyacrylamide thickener that is from at least 0.2%, 0.3%, 0.4%, or 0.5% up to 0.9%, 1.0%, 1.5%, or 2% by weight of the eyebrow primer, including all combinations and subranges thereof. In some embodiments, the total amount of polyacrylamide thickener is greater than 0.5% by weight and less than 1.0% by weight of the eyebrow primer.

Water-Dispersed Latex Polymer

In some embodiments, the eyebrow primer may include a water-dispersed latex polymer.

In some embodiments, the water-dispersed latex polymer is a polyacrylate latex polymer or its copolymer.

Non-limiting examples include ethylhexyl acrylate/hema copolymer (and) acrylates/diethylaminoethyl methacrylate/ethylhexyl acrylate copolymer (Syntran® PC 5775), styrene/acrylates/ammonium methacrylate copolymer (Syntran® 5760, Syntran® 5009, Syntran® PC5620), polyacrylate-21 (and) acrylates/dimethylaminoethyl methacrylate copolymer (Syntran® PC5100, Syntran® PC5776, Eudragit E 100, Jurymer ET-410C), styrene/acrylates/ammonium methacrylate copolymer (Syntran® 5009 CG), olefin/acrylate grafted polymer (and) sodium laureth sulfate (and C12-15 SEC-pareth 15 (Syntran® EX108), acrylates copolymer (Aculyn® 33A Polymer, Avalure® Ace 210/120/315 Acrylic Copolymer, Carbopol Aqua SF-1® Polymer, Daitosol® 500 AD, Coatex® Co 633, Eliclear® 380/700/4U, Eudragit® L 100, Joncryl® 85, Luviflex® Soft), acrylates/ethylhexyl acrylate copolymer (Daitosol® 5000SJ, Daitosol® 4000SJT, MJA PS34-21, SDP-001). The Syntran® polymers are commercially available from the supplier Interpolymer Corp.

In some embodiments, the latex polymer may be an acrylate latex polymer, in particular styrene/acrylate copolymers. Non-limiting examples of commercially available styrene/acrylate copolymers include, but are not limited to, DAITOSOL 5000 STY sold by Kobo Products, Inc.; JONCRYL® 77 sold by BASF; NEOCRYL BT-62 sold by Neoresins, Inc.; RHOPLEX™ P-376 and UCAR™ DL 432S sold by Dow Chemical Company; and YODOSOL GH41 and YODOSOL GH840 sold by AkzoNobel.

In some embodiments, the latex polymer is an anionic or bimodal acrylate latex polymer, such as Polyacrylate-15, Polyacrylate-21, Polyacrylate-17, Polyacrylate-18, Polyacrylate-19, a branched anionic acrylate copolymer Polyacrylate-2 Crosspolymer, Acrylates Crosspolymer-3, Polyacrylate-14, and those sold under the SYNTRAN series as commercially available from Interpolymer such as Acrylates Copolymer, Styrene/Acrylates/Ammonium Methacrylate Copolymer and Ammonium Acrylates Copolymer.

In some embodiments, the latex polymer is a polyurethane, such as polyurethane-1 or polyurethane-34.

In some embodiments, the total solid content of the latex (e.g., the total amount of the water-dispersed latex polymer) is at least 2% by weight of the eyebrow primer. In some embodiments, the total amount of the water-dispersed latex polymer is from at least 2%, 2.1%, 2.2%, or 2.3% up to 2.8%, 2.9%, or 3.0% by weight of the eyebrow primer, including all combinations and subranges thereof.

Water-Soluble Polymer

In some embodiments, the eyebrow primer may include a water-soluble polymer. In some embodiments, the water-soluble polymer may be a film-forming polymer.

As used herein, the term “water-soluble polymer” is intended to mean a polymer which, when introduced into water at a concentration equal to 1%, gives a macroscopically homogeneous solution of which the light transmittance, at a wavelength equal to 500 nm, through a sample 1 cm thick, is at least 10%.

As used here, the term “film-forming polymer” is intended to mean a polymer that is capable, by itself or in the presence of an auxiliary film-forming agent, of forming a macroscopically continuous deposit, and preferably a cohesive deposit, and even better still a deposit of which the cohesion and mechanical properties are such that said deposit can be isolated and manipulated individually, for example when said deposit is prepared by pouring onto a non-stick surface such as a teflonated or siliconated surface.

In some embodiments, a film-forming water-soluble polymer may include a (poly)vinylpyrrolidone (PVP) hydrophilic polymer.

In some embodiments, the (poly)vinylpyrrolidone hydrophilic polymer may be linear.

In some embodiments, the (poly)vinylpyrrolidone hydrophilic polymer may be chosen from statistical polymers, block copolymers, and a mixture thereof. As used herein, the term “block copolymer” is intended to mean a polymer comprising at least two different blocks and preferably at least three different blocks.

In some embodiments, the (poly)vinylpyrrolidone hydrophilic polymer may be chosen from: (poly)vinylpyrrolidone homopolymers; copolymers of (poly)vinylpyrrolidone/(poly)vinyl acetate, esters; copolymers of (poly)vinylpyrrolidone/(meth)acrylic, salts thereof; and a mixture thereof.

As (poly)vinylpyrrolidone homopolymers, mention may for example be made of the polyvinylpyrrolidone (2500 g/mol) sold under the trade name Kollidon 17 PF by the company BASF, the polyvinylpyrrolidone sold under the trade name Luviskol K 30 Powder by the company BASF or else sold under the trade name PVP K 30 L by the company ISP (Ashland), and the polyvinylpyrrolidone sold under the trade name PVP K 90 by the company ISP (Ashland). As (poly)vinylpyrrolidone/(poly)vinyl acetate copolymers, mention may for example be made of the vinylpyrrolidone/vinyl acetate (60/40) copolymer sold under the trade name Luviskol VA 64 Powder by the company BASF.

In some embodiments, the (poly)vinylpyrrolidone hydrophilic polymer suitable may have a weight-average molecular weight, Mw, ranging from 1500 to 500 000 g/mol.

In some embodiments, the total solid content of the water-(poly)vinylpyrrolidone hydrophilic polymer (e.g., the total amount of the water-soluble polymer itself) is at least 3% by weight of the eyebrow primer. In some embodiments, the total solid content of the water-(poly)vinylpyrrolidone hydrophilic polymer (e.g., the total amount of the water-soluble polymer itself) is less than 5% by weight of the eyebrow primer.

Polyhydric Alcohols

In some embodiments, the eyebrow primer may include one or more polyhydric alcohols.

In some embodiments, the polyhydric alcohols may comprise or consist of triols and diols.

In some embodiments, the polyhydric alcohols may include one or more C3-C6 polyhydric alcohols. The C3-C6 polyhydric alcohols may include glycerol (also referred to as glycerin), propylene glycol, butylene glycol, diethylene glycol, pentylene glycol, hexylene glycol, dipropylene glycol, sorbitol, or combinations thereof. In some embodiments, the C3-C6 polyhydric alcohols may consist of a glycerin and a plurality of glycols, such as glycols selected from propylene glycol, butylene glycol, diethylene glycol, pentylene glycol, hexylene glycol, and dipropylene glycol. In some embodiments, the total amount of glycerin is greater than the total amount of glycols. In some embodiments, the ratio of the total amount of glycerin to the total amount of glycols is 1.5:1 to 2:1.

In some embodiments, the polyhydric alcohols may include a C7-C12 polyhydric alcohol, such as caprylyl glycol.

In some embodiments, the eyebrow primer may include one, or at least one, C7-C12 polyhydric alcohol, and a plurality of C3-C6 polyhydric alcohols.

In some embodiments, the total amount of polyhydric alcohol is at least 2%, 2.5%, or 3%, up to 12%, 13%, 14%, or 15% by weight of the eyebrow primer, including all combinations and subranges thereof. In some embodiments, the total amount of C3-C6 polyhydric alcohol is at least 7%, 8%, or 9%, up to 11%, 12%, or 13% by weight of the eyebrow primer, including all combinations and subranges thereof. In some embodiments, the total amount of glycerin is at least 4%, 5%, or 5%, up to 8%, 9%, or 10% by weight of the eyebrow primer, including all combinations and subranges thereof. In some embodiments, the total amount of glycols is at least 1%, 2%, or 3%, up to 4%, 5%, or 6% by weight of the eyebrow primer, including all combinations and subranges thereof. In some embodiments, the eyebrow primer may be substantially free, of C7-C12 polyhydric alcohols.

Additional Surfactants or Co-Emulsifiers

In some embodiments, the eyebrow primer may include one or more additional surfactants or co-emulsifiers. In some embodiments, the one or more additional surfactants or co-emulsifiers is not a high HLB surfactant. In some embodiments, the additional surfactant may be a low HLB surfactant. As used herein, the term “low HLB surfactant” is intended to include any surfactant with a hydrophilic-lipophilic balance (HLB) less than 9.

In some embodiments, the additional surfactant or co-emulsifier may include a nonionic surfactant, such as a glyceryl fatty ester or sorbitan fatty ester.

In some embodiments, the additional surfactant or co-emulsifier may include a glyceryl fatty ester. Non-limiting examples of glyceryl esters include: glyceryl caprate, glyceryl caprylate, glyceryl oleate, glyceryl linoleate, glyceryl myristate, glyceryl capromyristate, glyceryl stearate, glyceryl hydroxy stearate, glyceryl isostearate, glyceryl ricinoleate, glyceryl dilaurate, glyceryl dioleate, glyceryl distearate, glyceryl mono/dicaprylate, glyceryl mono/dimyristate, glyceryl stearate palmitate, glyceryl tricaprate/caprylate, caprylic/capric diglycerylsuccinate, caprylic/capric glycerides, caprylic/capric/isostearic/adipic triglycerides, caprylic/capric/linoleic triglycerides, caprylic/capric triglycerides, caprylic/capric/stearic triglycerides, glyceryltrilaurate/stearate, glyceryldi/tripalmitostearate, glyceryldi/tritristearate, caprylic triglyceride, caprylic/capric/lauric triglycerides, glyceryl triheptanoate, glyceryl trioctanoate, glyceryl trilaurate, glyceryl trioleate, glyceryl tri stearate, glyceryltris-12-hydroxystearate, glyceryltriacetyl hydroxystearate, glyceryl triacetyl ricinoleate, glyceryl triisostearate, glyceryl tribehenate, and mixtures thereof.

In some embodiments, the additional surfactant or co-emulsifier may include a sorbitan ester. In some embodiments, the sorbitan ester may include sorbitan oleate, sorbitan stearate, sorbitan isostearate, or combinations thereof.

In some embodiments, the additional surfactant or co-emulsifier may include phosphate esters of alcohol ethoxylates. In some embodiments, the phosphate esters of alcohol ethoxylates may include trideceth-6 phosphate. In some embodiments, the eyebrow primer may be free or substantially free of phosphate esters of alcohol ethoxylates.

In some embodiments, the additional surfactant or co-emulsifier may include one or more anionic surfactants. Non-limiting examples of anionic surfactants include alkyl sulfates, alkyl ether sulfates, acyl isethionates, acyl glycinates, acyl taurates, acyl amino acids, acyl sarcosinates, sulfosuccinates, sulfonates, and a mixture thereof, wherein the alkyl and acyl groups of all these compounds comprise from 6 to 24 carbon atoms. In some embodiments, the anionic surfactant may be sodium laureth sulfate. In some embodiments, the eyebrow primer may be free, or substantially free, of sulfates. In some embodiments, the eyebrow primer may be free, or substantially free, of anionic surfactants.

In some embodiments, the total amount of additional surfactants or co-emulsifiers is from at least 0.5%, 1%, 1.5%, or 2% up to 3%, 3.5%, 4%, or 5% by weight of the eyebrow primer, including all combinations and subranges thereof. In some embodiments, the one or more additional surfactants or co-emulsifiers are present in a total amount of between 1% and 3% by weight of the eyebrow primer.

Water-Soluble Humectant or Solvent

In some embodiments, the eyebrow primer may include a water-soluble humectant, a water-soluble solvent, or both.

In some embodiments, the water-soluble solvent may be selected from lower alkyl alcohols and may be selected according to the desired skin feel to be delivered, and/or for delivering certain skin benefit agents. In some embodiments, the lower alkyl alcohols useful herein are monohydric alcohols having 1 to 6 carbons, more preferably ethanol and isopropanol.

In some embodiments, the water-soluble humectant may include a sugar (such as trehalose, maltose, glucose, or fructose), urea, sodium chondroitin sulfate, sodium hyaluronate, sodium adenosin phosphate, sodium lactate, pyrrolidone carbonate, cyclodextrin, or a combination thereof.

In some embodiments, the water-soluble humectant may include alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

In some embodiments, the eyebrow primer may be free or substantially free of water-soluble humectants or solvents. In some embodiments, the eyebrow primer may include from about 1% to about 20% of a water-soluble humectant and/or solvent. In some embodiments, the water-soluble humectant and/or solvent may be present in a total amount of between 5% and 10% by weight.

Chelating Agents and Preservatives

In some embodiments, the eyebrow filler may include one or more chelating agents and/or preservatives.

In some embodiments, the eyebrow filler may include a chelating agent. In some embodiments, the chelating agent may include ethylenediaminetetraacetic acid (EDTA) or a salt thereof. In some embodiments, the chelating agent may be disodium EDTA, trisodium EDTA, or tetrasodium EDTA.

In some embodiments, the eyebrow filler may include a preservative. Non-limiting examples of preservatives include benzoic acid and alkali metal and ammonium salts thereof (e.g., sodium benzoate), sorbic acid and alkali metal and ammonium salts thereof (e.g., potassium sorbate}, p-Anisic acid and alkali metal, and ammonium salts thereof, and salicylic acid and alkali metal and ammonium salts thereof. Other non-limiting examples of preservatives include ethanol, polyvinyl alcohol, phenoxyethanol, benzyl alcohol, salicylic acid, sodium benzoate, methyl paraben, propyl paraben, or a combination thereof

Oil

The eyebrow primer may include at least one oil.

As used herein, the term “oil” means a fatty substance that is liquid at room temperature (25° C.) and at atmospheric pressure. The disclosed oils should not fall into one of the other categories of materials described herein. In some embodiment, the at least one oil may include a non-volatile oil, a volatile oil, or a combination thereof.

In some embodiments, the eyebrow primer may include less than 5%, 4%, 3%, or 2% of oils. In some embodiments, the eyebrow primer may be substantially free of oils.

As used herein, the term “hydrocarbon-based oil” means an oil formed essentially from, or even constituted of, carbon and hydrogen atoms, and optionally oxygen and nitrogen atoms, and not containing any silicon or fluorine atoms. It may contain hydroxy, ester, ether, carboxylic acid, amine and/or amide groups.

As used herein, the term “nonvolatile oil” refers to an oil with a vapor pressure of less than 0.13 Pa.

As used herein, the term “silicone oil” means an oil comprising at least one silicon atom and notably at least one Si—O group.

As used herein, the term “volatile oil” refers to an oil (or non-aqueous medium) that can evaporate on contact with the skin in less than one hour, at room temperature and at atmospheric pressure. The volatile oil is a volatile cosmetic oil, which is liquid at room temperature, notably having a nonzero vapor pressure, at room temperature and at atmospheric pressure, in particular having a vapor pressure ranging from 0.13 Pa to 40 000 Pa (10-3 to 300 mmHg), preferably ranging from 1.3 Pa to 13,000 Pa (0.01 to 100 mmHg) and preferentially ranging from 1.3 Pa to 1300 Pa (0.01 to 10 mmHg).

Non-Volatile Oil

In some embodiments, the non-volatile oil may be a hydrocarbon-based oil, a silicone oil, or a mixture thereof

The Non-Volatile Hydrocarbon-Based Oils May Include:
hydrocarbon-based oils of plant origin, such as triglycerides consisting of fatty acid esters of glycerol, the fatty acids of which may have chain lengths ranging from C4 to C28, these fatty acids possibly being linear or branched, and saturated or unsaturated; these oils are in particular wheatgerm oil, sunflower oil, grapeseed oil, sesame oil, corn oil, apricot oil, castor oil, shea oil, avocado oil, olive oil, soybean oil, sweet almond oil, rapeseed oil, cottonseed oil, hazelnut oil, macadamia oil, jojoba oil, palm oil, alfalfa oil, poppy oil, pumpkin oil, marrow oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarineries Dubois or those sold under the names Miglyol 810®, 812® and 818® by the company Sasol;
synthetic ethers containing from 10 to 40 carbon atoms;
linear or branched hydrocarbons of mineral or synthetic origin other than the polymers according to the invention, such as petroleum jelly, polybutenes, polydecenes and squalane, and mixtures thereof;
synthetic esters such as the oils of formula R1COOR2 in which R1 represents the linear or branched fatty acid residue containing from 1 to 40 carbon atoms and R2 represents an in particular branched hydrocarbon-based chain containing from 1 to 40 carbon atoms, on condition that R1+R2 10, for instance purcellin oil (cetostearyl octanoate), isopropyl myristate, isopropyl palmitate, C12 to C15 alkyl benzoate, hexyl laurate, diisopropyl adipate, isononyl isononanoate, 2-ethylhexyl palmitate, isostearyl isostearate, alkyl or polyalkyl octanoates, hydroxylated esters such as isostearate lactate and diisostearyl malate; and pentaerythritol esters;
fatty alcohols that are liquid at ambient temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol; and
higher fatty acids such as oleic acid, linoleic acid or linolenic acid, and mixtures thereof.

Non-limiting examples of suitable non-volatile silicone oils that may be used in the composition in accordance with the invention may be non-volatile polydimethylsiloxanes (PDMSs), polydimethylsiloxanes comprising alkyl or alkoxy groups, that are pendent and/or at the end of a silicone chain, the groups each containing from 2 to 24 carbon atoms, phenyl silicones, such as phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

In some embodiments, the eyebrow primer may be free, or substantially free, of non-volatile oil.

Volatile Oil

The eyebrow primer may include at least one volatile oil.

In some embodiments, the volatile oil may be hydrocarbon-based. In some embodiments, the volatile hydrocarbon-based oil may be chosen from hydrocarbon-based oils containing from 7 to 16 carbon atoms.

In some embodiments, the eyebrow primer may include one or more volatile branched alkanes. In some embodiments, the volatile hydrocarbon-based oil containing from 7 to 16 carbon atoms may include C8-C16 branched alkanes, such as C8-C16 isoalkanes (also known as isoparaffins), isododecane, isodecane, isohexadecane and for example the oils sold under the trade names Isopar or Permethyl, C8-C16 branched esters such as isohexyl neopentanoate, and mixtures thereof. Preferably, the volatile hydrocarbon-based oil containing from 8 to 16 carbon atoms is chosen from isododecane, isodecane and isohexadecane, and mixtures thereof, and is in particular isododecane.

The eyebrow primer may contain one or more volatile linear alkanes. In some embodiments, a volatile linear alkane may be liquid at ambient temperature (about 25° C.) and at atmospheric pressure (760 mmHg). In some embodiments, volatile linear alkane may include a cosmetic linear alkane, which is capable of evaporating on contact with the skin in less than one hour, at ambient temperature (25° C.) and atmospheric pressure (760 mmHg), which is liquid at ambient temperature, in particular having an evaporation rate ranging from 0.01 to 15 mg/cm2/min, at ambient temperature (25° C.) and atmospheric pressure (760 mmHg).

In some embodiments, the linear alkanes, preferably of plant origin, may include from 7 to 15 carbon atoms, in particular from 9 to 14 carbon atoms and more particularly from 11 to 13 carbon atoms.

Non-limiting examples of linear alkanes include n-heptane (C7), n-octane (C8), n-nonane (C9), n-decane (C10), n-undecane (C11), n-dodecane (C12), n-tridecane (C13), n-tetradecane (C14) and n-pentadecane (C15), and mixtures thereof, and in particular the mixture of n-undecane (C11) and n-tridecane (C13) described in Example 1 of patent application WO 2008/155 059 by the company Cognis. Mention may also be made of n-dodecane (C12) and n-tetradecane (C14) sold by Sasol under the references, respectively, Parafol 12-97 and Parafol 14-97, and also mixtures thereof.

In some embodiments, the linear alkane may be used alone or as a mixture of at least two distinct alkanes that differ from each other by a carbon number of at least 1, and in particular a mixture of at least two linear alkanes comprising from 10 to 14 distinct carbon atoms that differ from each other by a carbon number of at least 2, and in particular a mixture of C11/C13 volatile linear alkanes or a mixture of C12/C14 linear alkanes, in particular an n-undecane/n-tridecane mixture.

In some embodiments, the eyebrow primer may include at least one volatile silicone oil or solvent that is compatible with cosmetic use.

Non-limiting examples of volatile silicone oils include cyclic polysiloxanes and linear polysiloxanes, and mixtures thereof. Volatile linear polysiloxanes that may be mentioned include hexamethyldisiloxane, octamethyltrisiloxane, decamethyltetrasiloxane, tetradecamethylhexasiloxane and hexadecamethylheptasiloxane. Volatile cyclic polysiloxanes that may be mentioned include hexamethylcyclotrisiloxane, octamethylcyclotetrasiloxane, decamethylcyclopentasiloxane and dodecamethylcyclohexasiloxane.

In some embodiments, the eyebrow primer may be free, or substantially free, of volatile oil.

Acid Thickener

In some embodiments, the eyebrow primer may be free of an acid thickener. As used herein, the term “acid thickener” refers to acid-based thickening agents that require neutralization to provide the desired thickening property. Non-limiting examples of such acid thickeners include polyacrylic acid/alkyl acrylate copolymers, preferably modified or unmodified carboxyvinyl polymers; the copolymers most particularly preferred according to the present invention are acrylate/C₁₀-C₃₀-alkylacrylate copolymers (INCI name: Acrylates/C₁₀₋₃₀ Alkyl acrylate Crosspolymer) such as the products sold by the company Lubrizol under the trade names Pemulen TR1, Pemulen TR2, Carbopol 1382 and Carbopol ETD 2020, and even more preferentially Pemulen TR-2.

Other Clays

Talc is a clay mineral. However, in some embodiments, the eyebrow primer may be free of other clays. In some embodiments, the other clays may include lipophilic and/or hydrophilic clays.

The term “hydrophilic clay” means a clay that is capable of swelling in water; this clay swells in water and forms after hydration a colloidal dispersion.

These clays are products that are already well known per se, which are described, for example, in the book “Minéralogie des argiles”, S. Caillère, S. Hénin, M. Rautureau, 2^{nd} edition 1982, Masson, the teaching of which is included herein by way of reference. Clays are silicates containing a cation that may be chosen from calcium, magnesium, aluminium, sodium, potassium and lithium cations, and mixtures thereof. Examples of such products that may be mentioned include clays of the smectite family such as montmorillonites, hectorites, bentonites, beidellites and saponites, and also of the family of vermiculites, stevensite and chlorites.

These clays may be of natural or synthetic origin. Hydrophilic clays that may be mentioned include kaolinite; smectite products such as saponites, hectorites, montmorillonites, bentonites and beidellite; synthetic hectorites (also known as laponites), for instance the products sold by the company Laporte under the names Laponite XLG, Laponite RD and Laponite RDS (these products are sodium magnesium silicates and in particular sodium lithium magnesium silicates); bentonites, for instance the product sold under the name Bentone HC by the company Rheox; magnesium aluminium silicates, especially hydrated, for instance the products sold by the Vanderbilt Company under the names Veegum Ultra, Veegum HS and Veegum DGT, or calcium silicates, and especially the product in synthetic form sold by the company under the name Micro-cel C.

The term “lipophilic clay” means a clay that is capable of swelling in a lipophilic medium; this clay swells in the medium and thus forms a colloidal dispersion.

Examples of lipophilic clays that may be mentioned include modified clays such as modified magnesium silicate (Bentone Gel VS38 from Rheox), and hectorites modified with a C₁₀ to C₂₂ fatty-acid ammonium chloride, for instance hectorite modified with distearyldimethylammonium chloride (CTFA name: disteardimonium hectorite) sold under the name Bentone 38 CE by the company Rheox or Bentone 38V® by the company Elementis.

Pigments and Other Colorants

In some embodiments, the composition may include a colorant. In some embodiments, the colorant may be a pigment, a pearlescent agent, or a combination thereof. In some embodiments, the composition can advantageously comprise at least one colorant chosen from pigments and/or pearlescent agents.

In some embodiments, the eyebrow primer may include less than 5%, 4%, 3%, or 2% of colorant. In some embodiments, the eyebrow primer may be free, or substantially free, of colorant.

In some embodiments, the eyebrow primer may include less than 5%, 4%, 3%, or 2% of pigment. In some embodiments, the eyebrow primer may be free, or substantially free, of pigment.

Pigments

As used herein, the term “pigment” refers to white or colored and inorganic (mineral) or organic particles which are insoluble in the lipophilic phase(s) and which are intended to color and/or opacify the composition and/or the deposited layer produced with the composition.

The pigments may be chosen from mineral pigments, organic pigments and composite pigments (i.e., pigments based on mineral and/or organic materials).

The pigments can be chosen from mineral pigments, in particular monochromatic pigments, organic lakes, pearlescent agents and goniochromatic pigments.

If the composition comprises them, their content varies from 0.1% to 15% by weight, with respect to the weight of the composition, and preferably from 0.5% to 12% by weight, with respect to the weight of the composition.

The mineral pigments can be chosen from metal oxide pigments, chromium oxides, iron oxides (black, yellow, red), titanium dioxide, zinc oxides, cerium oxides, zirconium oxides, chromium hydrate, manganese violet, Prussian blue, ultramarine blue, ferric blue, metal powders, such as aluminum powders or copper powder, and their mixtures.

Organic lakes are organic pigments, formed of a dye attached to a substrate. The lakes, which are also known as organic pigments, can be chosen from the materials below and their mixtures:
cochineal carmine;
organic pigments of azo dyes, anthraquinone dyes, indigoid dyes, xanthene dyes, pyrene dyes, quinoline dyes, triphenylmethane dyes or fluoran dyes.

Mention may in particular be made, among the organic pigments, of those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5 or FD&C Yellow No. 6;
the organic lakes can be insoluble sodium, potassium, calcium, barium, aluminum, zirconium, strontium or titanium salts of acid dyes, such as azo, anthraquinone, indigoid, xanthene, pyrene, quinoline, triphenylmethane or fluoran dyes, these dyes possibly comprising at least one carboxylic or sulfonic acid group.

The organic lakes can also be supported by an organic support, such as rosin or aluminum benzoate, for example.

Mention may in particular be made, among the organic lakes, of those known under the following names: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake or FD&C Yellow No. 6 Aluminum lake.

Mention may also be made of liposoluble dyes, such as, for example, Sudan Red, DC Red 17, DC Green 6, β-carotene, soybean oil, Sudan Brown, DC Yellow 11, DC Violet 2, DC Orange 5 or quinoline yellow.

The chemical substances corresponding to each of the organic colourants cited above are mentioned in the publication “International Cosmetic Ingredient Dictionary and Handbook”, 1997 edition, pages 371 to 386 and 524 to 528, published by The Cosmetic, Toiletries and Fragrance Association, the content of which is incorporated into the present patent application by reference.

The pigments may also have been subjected to a hydrophobic treatment.

The hydrophobic treatment agent can be chosen from silicones, such as methicones, dimethicones, alkoxysilanes and perfluoroalkylsilanes; fatty acids, such as stearic acid; metal soaps, such as aluminum dimyristate, the aluminum salt of hydrogenated tallow glutamate, perfluoroalkyl phosphates, perfluoroalkylsilanes, perfluoroalkylsilazanes, poly(hexafluoropropylene oxide)s, polyorganosiloxanes comprising perfluoroalkyl perfluoropolyether groups and amino acids; N-acylated amino acids or their salts; lecithin, isopropyl triisostearyl titanate, and their mixtures.

The N-acylated amino acids can comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be aluminum, magnesium, calcium, zirconium, zinc, sodium or potassium salts. The amino acid can, for example, be lysine, glutamic acid or alanine.

The term “alkyl” cited in the abovementioned compounds denotes in particular an alkyl group having from 1 to 30 carbon atoms and preferably having from 5 to 16 carbon atoms.

Hydrophobic treated pigments are described in particular in Application EP-A-1 086 683.

Pearlescent Agents

As used herein, the term “pearlescent agent” refers to colored particles of any shape, which are or are not iridescent, and which may be of natural or synthetic origin, and which exhibit a color effect via optical interference.

Mention may be made, as examples of pearlescent agents, of pearlescent pigments, such as titanium oxide-coated mica covered with an iron oxide, mica covered with bismuth oxychloride, titanium oxide-coated mica covered with chromium oxide, titanium oxide-coated mica covered with an organic dye, in particular of the abovementioned type, and also pearlescent pigments based on bismuth oxychloride.

They can also be mica particles, at the surface of which are superimposed at least two successive layers of metal oxides and/or of organic colorants.

The pearlescent agents can more particularly have a yellow, pink, red, bronze, orangey, brown, gold and/or coppery color or glint.

Mention may be made, by way of illustration of the pearlescent agents which can be introduced as interference pigment into the first composition, of gold-colored pearlescent agents sold in particular by BASF under the name Brilliant Gold 212G (Timica), Gold 222C (Cloisonne), Sparkle Gold (Timica) and Monarch Gold 233X (Cloisonne); bronze pearlescent agents sold in particular by Merck under the names Bronze Fine (17384) (Colorona) and Bronze (17353) (Colorona) and by BASF under the name Super Bronze (Cloisonne); orange pearlescent agents sold in particular by BASF under the name Orange 363C (Cloisonne) and by Merck under the names Passion Orange (Colorona) and Matte Orange (17449) (Microna); brown-colored pearlescent agents sold in particular by BASF under the names Nu-Antique Copper 340XB (Cloisonne) and Brown CL4509 (Chroma-lite); pearlescent agents with a copper glint sold in particular by BASF under the name Copper 340A (Timica); pearlescent agents with a red glint sold in particular by Merck under the name Sienna Fine (17386) (Colorona); pearlescent agents with a yellow glint sold in particular by BASF under the name Yellow (4502) (Chroma-lite); red-colored pearlescent agents with a gold glint sold in particular by BASF under the name Sunstone G012 (Gemtone); pink pearlescent agents sold in particular by BASF under the name Tan Opal G005 (Gemtone); black pearlescent agents with a gold glint sold in particular by BASF under the name Nu-Antique Bronze 240 AB (Timica); blue pearlescent agents sold in particular by Merck under the name Matte Blue (17433) (Microna); white pearlescent agents with a silvery glint sold in particular by Merck under the name Xirona Silver; and golden green pinkish orangey pearlescent agents sold in particular by Merck under the name Indian Summer (Xirona); and their mixtures.

Goniochromatic Pigments

As used herein, the term “goniochromatic pigment” refers to a pigment which makes it possible to obtain, when the composition is spread over a substrate, a color distance in the a*b* plane of the CIE 1976 colorimetric space which corresponds to a variation Dh° in the angle of hue h° of at least 20° when the angle of observation is varied with respect to the normal by between 0° and 80°, for an angle of incidence of the light of 45°.

The color distance can be measured, for example, using a spectrogonioreflectometer of the Instrument Systems brand and with the GON 360 Goniometer reference, after the composition has been spread in the fluid state with a thickness of 300 μm using an automatic spreader over a contrast chart of the Erichsen brand and with the Type 24/5 reference, the measurement being carried out on the black background of the chart.

The goniochromatic pigment can be chosen, for example, from multilayer interference structures and liquid crystal coloring agents.

In the case of a multilayer structure, the latter can comprise, for example, at least two layers, each layer being produced, for example, from at least one material chosen from the group consisting of the following materials: MgF2, CeF3, ZnS, ZnSe, Si, SiO2, Ge, Te, Fe2O3, Pt, Va, Al2O3, MgO, Y2O3, S2O3, SiO, HfO2, ZrO2, CeO2, Nb2O5, Ta2O5, TiO2, Ag, Al, Au, Cu, Rb, Ti, Ta, W, Zn, MoS2, cryolite, alloys, polymers and their combinations.

The multilayer structure may or may not exhibit, with respect to a central layer, a symmetry with regard to the chemical nature of the stacked layers.

Different effects are obtained according to the thickness and the nature of the various layers.

Examples of symmetrical multilayer interference structures are, for example, the following structures: Fe2O3/SiO2/Fe2O3/SiO2/Fe2O3, a pigment having this structure being sold under the name Sicopearl by BASF; MoS2/SiO2/mica-oxide/SiO2/MoS2; Fe2O3/SiO2/mica-oxide/SiO2/Fe2O3; TiO2/SiO2/TiO2 and TiO2/Al2O3/TiO2, pigments having these structures being sold under the name Xirona by Merck.

The liquid crystal coloring agents comprise, for example, silicones or cellulose ethers to which mesomorphic groups are grafted. Use may be made, as liquid crystal goniochromatic particles, for example, of those sold by Chenix and of those sold under the name Helicone® HC by Wacker.

Use may also be made, as goniochromatic pigment, of certain pearlescent agents, effect pigments on a synthetic substrate, in particular a substrate of alumina, silica, borosilicate, iron oxide or aluminum type, or interference glitter resulting from a polyterephthalate film.

Mention may in particular be made, as non-limiting examples of goniochromatic pigments, alone or as mixtures, of the goniochromatic pigments SunShine® sold by Sun Chemical, Cosmicolor Celeste® from Toyo Aluminum K.K., Xirona® from Merck and Reflecks Multidimensions® from BASF.

Dyes

The composition according to the invention may also comprise one or more dyes.

Among the liposoluble dyes, mention may be made especially of fluoran dyes such as, for example, red Sudan, FDC Red 4, DC Red 17, Red 21, Red 27, DC Green 6, Sudan brown, Yellow 10, DC Yellow 11, DC Violet 2, DC Orange 4, DC Orange 5, Yellow quinoline, or mixtures thereof.

Also disclosed is a method for priming and/or styling eyebrows. The method may include first applying an embodiment of an eyebrow primer as disclosed herein to an eyebrow, and then applying an eyebrow makeup composition over the eyebrow primer. In some embodiments, the eyebrow makeup composition is a composition for a brow pencil. In some embodiments, the eyebrow makeup composition is a powder. In some embodiments, the eyebrow makeup composition is a hot pour applied with a brush.

### Examples

The exemplary formulas shown in Table 1, below, were produced by combining the necessary materials, heating to around 70-75° C., mixing until homogenous, and cooling to room temperature.

TABLE 1
(Example Formulas, with two different latex polymers)

| Material | Ex 1 | Ex 2 |
|---|---|---|
| Water | 60-65 | 60-65 |
| Transparent Filler | 8.0-10.0 | 8.0-10.0 |
| High HLB surfactants | 6.0-8.0 | 6.0-8.0 |
| Polyacrylamide Thickener | 0.5-0.99 | 0.5-0.99 |
| (Acrylamide/Sodium | | |
| Acryloyldimethyltaurate Copolymer) | | |
| Latex Polymer (Styrene/ | 2.0-3.0 | — |
| Acrylates/Ammonium | | |
| Methacrylate Copolymer) | | |
| Latex Polymer (Polyacrylate-21) | — | 2.0-3.0 |
| Water-soluble polymer | 3-3.5 | 3-3.5 |
| Polyhydric Alcohol (Glycerin) | 6.0-8.0 | 6.0-8.0 |
| Polyhydric Alcohol (Glycols) | 3-3.5 | 3-3.5 |
| Add′l Surfactant (Low | 1.0-2.0 | 1.0-2.0 |
| HLB non-ionic surfactants) | | |
| Add′l Surfactant (Anionic surfactant) | 0.001-0.1 | 0.001-0.1 |
| Chelating Agent | 0.001-0.1 | 0.001-0.1 |
| Preservative | 0.5-0.89 | 0.5-0.89 |
| Oil | 0.25-0.75 | 0.25-0.75 |

The products were examined by a panel of experts, using in vivo testing and subjective analyses, and compared to a commercially available, competitive brow gel product. The products were first examined for a range of features relating to application and product, each feature being rated on a scale of 0-100%. Two features were the applicator's deposition of a high amount of product, and the applicator's deposition of too much product. Here, the exemplary formula provided a statistically significant lower rating of both (5% exemplary vs. 57% competitive for high deposit, and 0% exemplary vs. 48% competitive for depositing to much product), indicating a surprisingly more controlled user application experience. The exemplary formula was also compared for visibility on the brows, and again, the exemplary formula provided a statistically significant lower rating (20% exemplary vs. 56% competitive), indicating that, surprisingly, the exemplary formulas are far less visible on the brow.

The products were then examined for a range of features relating to eyebrow look and feel immediately after application. Two features considered were whether the product causes the eyebrows to either appear clumped or appear flattened. Here, the exemplary formula provided a statistically significant lower rating for both (0% exemplary vs. 22% competitive for clumping, 10% exemplary vs 45% competitive for flattened), indicating a surprisingly more aesthetic appearance.

The products were finally examined for a range of features relating to eyebrow look and feel 8 hours after application. One feature considered was whether the product causes the eyebrows to either appear flattened. Here, the exemplary formula provided a statistically significant lower rating (10% exemplary vs 40% competitive for flattened), indicating a more aesthetic appearance even after 8 hours of wear.

Those skilled in the art will recognize or be able to ascertain using no more than routine experimentation, many equivalents to the specific embodiments of the invention described herein. Such equivalents are intended to be encompassed by the following claims.

1. An eyebrow primer, comprising:
at least 5% by weight of a transparent filler;
a polyacrylamide thickener;
at least 2% by weight of one or more high HLB surfactants;
a water-dispersed latex polymer;
wherein the eyebrow primer is substantially free of oils; and
wherein the transparent filler consists of talc, mica, powders of polyamide, poly-β-3-alanine powders, polyethylene powders, polyurethane powders, lauroyl lysine; starch, boron nitride, polymeric hollow microspheres, silicone resin powders, hollow silica microspheres, glass microcapsules, ceramic microcapsules, or a combination thereof.

2. The eyebrow primer according to claim 1, wherein the transparent filler consists of talc, mica, or a combination thereof.

3. The eyebrow primer according to claim 2, wherein the transparent filler consists of talc.

4. The eyebrow primer according to claim 1, wherein the transparent filler comprises between 5% and 10% by weight of talc.

5. The eyebrow primer according to claim 1, wherein the one or more high HLB surfactants comprises ceteareth-25.

6. The eyebrow primer according to claim 1, wherein the polyacrylamide thickener is present in an amount of at least 0.5% by weight.

7. The eyebrow primer according to claim 1, wherein the water-dispersed latex polymer is present in an amount of at least 2% by weight.

8. The eyebrow primer according to claim 1, further comprising a water-soluble polymer.

9. The eyebrow primer according to claim 8, wherein the water-soluble polymer comprises polyvinylpyrrolidone (PVP).

10. The eyebrow primer according to claim 8, wherein water-soluble polymer is present in an amount of at least 3% by weight.

11. The eyebrow primer according to claim 1, further comprising one or more polyhydric alcohols.

12. The eyebrow primer according to claim 11, wherein the one or more polyhydric alcohols comprise one or more C3-C6 polyhydric alcohols.

13. The eyebrow primer according to claim 12, wherein the one or more polyhydric alcohols comprise one or more C7-C12 polyhydric alcohols.

14. The eyebrow primer according to claim 8, wherein the water-soluble humectant or solvent is present in a total amount of 7%-13% by weight.

15. The eyebrow primer according to claim 11, wherein one or more polyhydric alcohols consists of glycerin and a plurality of glycols.

16. The eyebrow primer according to claim 1, further comprising one or more additional surfactants or co-emulsifiers, the one or more additional surfactants or co-emulsifiers not being a high HLB surfactant.

17. The eyebrow primer according to claim 14, wherein the one or more additional surfactants or co-emulsifiers are present in an amount of between 1% and 3% by weight.

18. The eyebrow primer according to claim 1, wherein the eyebrow primer is free of other clays.

19. The eyebrow primer according to claim 18, wherein the eyebrow primer is free of pigments.

20. A method for priming eyebrows, comprising:
applying an eyebrow primer according to claim 1 to an eyebrow; and
applying an eyebrow makeup composition over the eyebrow primer.

21. The eyebrow primer according to claim 1, wherein the transparent filler is present in an amount of at least 10% by weight of the composition.
